(19) United States
(12) Patent Application Publication
(10) Pub. No.: US 2012/0269806 A1
(43) Pub. Date: Oct. 25, 2012

(54) METHODS OF INDUCING TOLERANCE

(75) Inventor: Megan A. Sykes, Bronx, NY (US)

(73) Assignee: The General Hospital Corporation, Boston, MA (US)

(21) Appl. No.: 12/674,353

(22) PCT Filed: Aug. 21, 2008
(86) PCT No.: PCT/US08/73932

§ 371 Date: Apr. 25, 2012

Related U.S. Application Data
(60) Provisional application No. 60/965,578, filed on Aug. 21, 2007.

(51) Int. Cl. A61K 38/02; A61K 38/13; A61P 37/06; A61M 37/00; A61P 1/16; A61P 13/12; A61N 5/10; A61K 39/395; A61P 9/00

(52) U.S. Cl. 424/134.1; 424/278.1; 424/173.1; 600/1

(57) ABSTRACT

Methods of inducing tolerance, and promoting graft acceptance, are described herein. The methods include administering to a recipient hematopoietic stem cells and an agonist of Programmed Death 1 (PD-1).

### CROSS-REFERENCE TO RELATED APPLICATIONS

This application claims the benefit of priority of U.S. Ser. No. 60/965,578, filed Aug. 21, 2007, the contents of which are hereby incorporated by reference in their entirety for all purposes.

### GOVERNMENT SUPPORT

The United States Government has provided grant support utilized in the development of the present invention. In particular, NIH grant R01 HL49915 has supported development of this invention. The United States Government may have certain rights in the invention.

### BACKGROUND OF THE INVENTION

The balance between stimulatory and inhibitory signals following T-cell receptor (TCR) engagement critically regulates the outcome of the immune response and can lead to T cell activation or tolerance. Costimulation blockade and activation of inhibitory pathways are potential approaches to controlling T cell reactivity in autoimmunity and transplantation. CD8 T cells have been shown to be more resistant to costimulation blockade than CD4 T cells (Jones et al., J. Immunol. 2000; 165:1111-1118). Indeed, in a model of allogeneic bone marrow transplantation (BMT), a conditioning regimen involving anti-CD154 and 3Gy total body irradiation (TBI) on Day 0 led to tolerance of donor-reactive CD4 T cells but not CD8 T cells (Ito et al., J. Immunol. 2001; 166:2981). Pathways involved in CD4 and CD8 tolerance with BMT are linked but different. While blocking CD154 tolerizes alloreactive CD4 T cells by inducing anergy, pathways involved in CD8 tolerance, and effective means for inducing CD8 tolerance, have not been identified.

### SUMMARY OF THE INVENTION

The present invention provides methods of inducing tolerance and promoting graft acceptance. The invention is based, in part, on the discovery that aspects of T cell tolerance are dependent on the Programmed Cell Death-1 (PD-1)/Programmed Cell Death-1 Ligand (PD-L1) pathway, and that manipulating this pathway induces T cell tolerance, e.g., in tolerance-inducing regimens that employ hematopoietic stem cell transplantation. In particular, it has been discovered that CD8 T cell tolerance is dependent on the PD-1/PD-L1 pathway. Agonizing PD-1 can induce tolerance and overcome CD8 T cell resistance. CD8 T cell tolerance is particularly difficult to achieve in bone marrow transplantation regimens employing costimulatory blockade. PD-1 agonists are useful for overcoming CD8 T cell-mediated reactivity in these, and other, regimens. The methods herein induce T cell tolerance to permit donor-specific immunological non-responsiveness, promote graft acceptance, and can eliminate the need for long-term immunosuppression in transplant recipients.

Accordingly, in one aspect, the invention features a method of inducing tolerance, and a method of promoting acceptance, by a recipient mammal, of a graft from a donor mammal. The method includes administering to the recipient, a Programmed Cell Death 1 (PD-1) agonist; and introducing into the recipient mammal, hematopoietic stem cells.

In some embodiments, a PD-1 agonist includes a Programmed Cell Death 1 Ligand 1 (PD-L1) polypeptide, or a biologically active fragment thereof (e.g., a fragment which binds to PD-1 and induces one more PD-1 associated activities). In some embodiments, a PD-1 agonist includes a soluble PD-L1 polypeptide (e.g., a PD-L1 polypeptide containing the extracellular domain, e.g., PD-L1-Ig).

In some embodiments, a PD-1 agonist includes a soluble Programmed Cell Death 1 Ligand 2 (PD-L2) polypeptide, or a biologically active fragment thereof (e.g., a fragment which binds to PD-1 and induces one more PD-1 associated activities). In some embodiments, a PD-1 agonist includes a soluble PD-L2 polypeptide (e.g., a PD-L2 polypeptide containing the extracellular domain, e.g., PD-L2-Ig). More than one type of PD-1 agonist can be used in the method.

In the methods of promoting acceptance, a PD-1 agonist can be administered prior to (e.g., immediately prior to), at the same time as, and/or after administering the hematopoietic stem cells. For example, a PD-1 agonist can be administered just prior to, and for 1, 2, 3, 4, 5, 6, 7 or more days after administering the hematopoietic stem cells.

The method can further include administering an inhibitor of a costimulatory pathway (e.g., an inhibitor of CD40-CD154 interaction, an inhibitor of CD28-B7 interaction). An inhibitor of a costimulatory pathway can administered prior to (e.g., immediately prior to), and/or at the same time as, the hematopoietic stem cells. An inhibitor of a costimulatory pathway can be administered after the hematopoeitic stem cells. In some embodiments, an inhibitor of CD40-CD154 interaction is administered, and the inhibitor comprises an anti-CD154 antibody, or an anti-CD40 antibody. In some embodiments, an inhibitor of CD28-B7 interaction (e.g., an anti-CD28, or anti-B7 antibody) is administered. In some embodiments, an inhibitor of a costimulatory pathway is administered which is CTLA4-Ig.

The method of promoting acceptance of a graft can further include implanting a graft (e.g., a heart, kidney, liver, lung, skin, pancreas, bone, endocrine, or other type of graft) in the recipient mammal. The graft can be implanted prior to, at the same time as, or after, administering the hematopoietic stem cells. In some embodiments, a PD-1 agonist is administered prior to, simultaneously with, or after the graft is implanted.

The donor mammal can be the same species as the recipient mammal. In some embodiments, the donor and recipient are matched at MHC alleles. In other embodiments, the donor and recipient are MHC-mismatched. The donor and recipient can be related (e.g., siblings, cousins), or can be unrelated. In some embodiments, the donor and recipient are humans.

In other embodiments, the donor mammal is a different species than the recipient. In some embodiments, the donor mammal is a swine (e.g., a miniature swine, e.g., an α-1,3-galactosyltransferase deficient swine), and the recipient mammal is a primate, e.g., a human.

The method can include administering an anti-T cell antibody to the recipient mammal. The anti-T cell antibody can include an anti-CD2 antibody. In some embodiments, the anti-T cell antibody includes an anti-CD3, anti-CD4, or anti-CD8 antibody. In some embodiments, the anti-T cell antibody includes anti-thymocyte globulin (ATG).

The method can include administration of an immunosuppressive agent to the recipient mammal (e.g., cyclosporine A, FK506, rapamycin). In some embodiments, the recipient receives a non-chronic course of the immunosuppressive agent. In some embodiments, the method permits use of a reduced dose (e.g., use of an amount of the immunosuppressive agent which would otherwise be insufficient to prevent graft rejection).

The recipient can be prepared for hematopoeitic cell transplantation by treatment with irradation or chemotherapy (e.g., to create hematopoietic space). In some embodiments, the method comprises administering non-myeloablative irradiation or a non-myeloablative chemotherapeutic treatment (e.g., cyclophosphamide) to the recipient mammal, prior to administering the hematopoietic stem cells. In some embodiments, the amount of hematopoeitic stem cells introduced into the recipient is sufficient to induce chimerism (transient or long term) in the recipient.

In some embodiments, the method of introducing hematopoietic stem cells and a PD-1 agonist promotes acceptance of the hematopoietic stem cells. In some embodiments, the method of introducing hematopoietic stem cells reduces graft versus host disease (GVHD).

In embodiments in which a graft separate from the hematopoietic stem cells is administered, the graft and the hematopoietic stem cells are from the same donor mammal. In some embodiments, the graft and the hematopoietic stem cells can be from different donor mammals.

The details of one or more embodiments of the invention are set forth in the accompanying drawings and the description below. Other features, objects, and advantages of the invention will be apparent from the description and drawings, and from the claims. All cited patents, patent applications, and references (including references to public sequence database entries) are incorporated by reference in their entireties for all purposes. U.S. Ser. No. 60/965,578 is incorporated by reference in its entirety for all purposes.

### BRIEF DESCRIPTION OF THE DRAWINGS

### FIG. 1. Generation of 2C/B6 Syngeneic Chimeras

(FIG. 1A) B6 mice received 3Gy TBI and 5×10⁶ syngeneic 2C BMCs. Seven weeks later, the presence of 2C cells among peripheral blood CD8 cells of the recipient mice was analyzed by FCM analysis using anti-clonotypic mAb 1B2. Histograms show 1B2 staining on gated CD8⁺ WBC. These 2C/B6 syngeneic chimeras then received 3Gy TBI on Day −1 and allo-BMT on Day 0. 2C/B6 mice were transplanted with either L^{d+} (B10.A) or L^{d-} (A.SW or B10.S) BM and injected with anti-CD154 on Day 0 or received L^{d+} (B10.A) BM without anti-CD154 (MR1). (FIG. 1B) Normalized mean (±SEM) percentage of 2C cells among splenic CD8 cells at 4 and 7 days after allogeneic BMT. A value of 100% was given to the percentage of 2C CD8⁺ T cells in the blood 1 week prior to allogeneic BMT and the percentage of 2C CD8⁺ T cells in the spleen on Days 4 and 7 was normalized to this value. Statistical analyses were performed with a Mann-Whitney U test to compare the “L^{d+} group” with the “L^{d-} group” or the “L^{d+} group” with the “L^{d+} no MR1 group”: ** p<0.005, *** p<0.0005, N.S. Not significant. Two experiments are shown for the Day 4 analysis (n=2-5 animals/group/experiment) and 3 experiments are shown for the Day 7 analysis (n=3-5 animals/group/experiment). (FIG. 1C) Time course of 2C deletion in peripheral WBC. A value of 1 was given to the percentage of 2C CD8⁺ T cells in the blood 1 week prior to allogeneic BMT. The percentage of 2C+CD8+ cells among WBC CD8 cells was then analyzed every 2 weeks and normalized to the value before BMT. The mean (±SEM) is presented. □: recipients of L^{d+} (B10.A); 0: recipients of L^{d-} (B10.S); Δ: recipients L^{d+} (B10.A) without anti-CD154.

FIG. 2: Up-Regulation of Activation Markers on CD8 Cells from Chimeric and Rejecting Mice Upon Specific Stimulation In Vivo.

(FIG. 3A) 2C/B6 mice were prepared 7 weeks before allo-BMT and received 3Gy TBI on Day −1 and allo-BMT on Day 0. 2C/B6 mice were transplanted with either L^{d+} (B10.A; H-2^{a}) or L^{d-} (A.SW or B10.S; H-2^{s}) BM and injected with anti-CD154 on Day 0 or received L^{d+} (B10.A) BM without anti-CD154 (MR1). Activation markers were assessed on Days 4 and 7 on 2C CD8⁺ and non-2C CD8⁺ splenocytes by FCM. Statistical analyses were performed with a Mann-Whitney U test to compare the “L^{d+} group” with the “L^{d-} group” or the “L^{d+} group” with “L^{d+} non MR1 group”: * p<0.05, ** p<0.01, *** p<0.001, N.S. Not significant. Two experiments are shown for the Day 4 analysis (n=2-5 animals/group/experiment) and 3 experiments are shown for the Day 7 analysis (n=3-5 animals/group/experiment). (FIG. 3B) Expression of activation/memory markers on 2C CD8⁺ and non-2C CD8⁺ splenocytes by FCM is analyzed in 2C/B6 mice that did not received conditioning or allogeneic BMT. The mean percentage of cells expressing the indicated markers 4 days after L^{d+} BMT (with anti-CD154) in another experiment is represented by: ▪. One experiment is shown with 10 animals.

### FIG. 3: Early Tolerance of CD8 T Cells.

Cytolytic capacity of tolerized donor-reactive CD8 T cells was analyzed with a ⁵¹Cr release assay following a 5 day restimulation in vitro. 2C/B6 mice were prepared 7 weeks before allo-BMT and received 3Gy TBI on Day −1 and allo-BMT on Day 0. 2C/B6 mice were transplanted with either L^{d+} (B10.A) (FIG. 3A) or L^{d-} (A.SW or B10.S) BM (FIG. 3B) and injected with anti-CD154 on Day 0 or received L^{d+} (B10.A) BM without anti-CD154 (MR1) (FIG. 3C). Four days after BMT splenocytes were tested against donor (B10.A) stimulator and target cells (▪) and against third party (B10.RIII) stimulator and target cells (□). One representative experiment out of 3 is shown (n=2-3 animals per group).

### FIG. 4: Requirement for the PD-1/PD-L1 Pathway for CD8 but not CD4 T Cell Tolerance.

(FIG. 4A) C57BL/6 or PD-1 KO mice (on a C57BL/6 background), CD8 depleted or not, received 20 to 25×10⁶ B10.A BM cells with anti-CD154 and 3Gy TBI. Incidence of chimerism is shown for the B cell lineage 6 weeks after BMT and is representative of all lineages analyzed. Multilineage chimerism 6 weeks post-BMT is shown in Table 1. One representative experiment out of 3 total is shown (n=5-8 animals/group/experiment). (FIG. 4B) B6 mice, CD8 depleted or not, received 20 to 25×10⁶ B10.A BM cells with PD-1 and PD-L1 blocking mAbs, anti-PD-L1 alone, anti-PD-1 alone or control irrelevant IgG2a and/or IgG2b mAbs, anti-CD154 and 3Gy TBI. Incidence of chimerism is shown for the B cell lineage 6 weeks after BMT and is representative of all lineages analyzed. Multilineage chimerism 6 weeks post-BMT is shown in Table 2. Incidence of chimerism was similar in control group treated with both irrelevant mAbs or with each irrelevant mAb alone. One representative experiment out of 2 total is shown (n=5-8 animals/group/experiment).

### FIG. 5: Modulation of PD-L1 Expression on B Cells and Dendritic Cells.

C57BL/6 mice received 3Gy TBI on D-1 followed by 20−25×10⁶ B10.A BM cells with or without anti-CD154 (MR1) on Day 0 (FIG. 5A); or anti-CD154 with or without CD4 depletion (FIG. 5B). Four days later, dendritic cells (CD11c+) and B cells (CD19+) were extracted from the spleen as described in the Materials and Methods section and then stained and analyzed by FCM. Dotted line represents a normal control mouse. Statistical analyses were performed with a Mann-Whitney U test * p<0.05, ** p<0.005. One experiment is shown for FIG. 5A and one experiment is shown for FIG. 5B. Each symbol represents an individual animal

### FIG. 6: Cell-Intrinsic Requirement for PD-1 on CD8 T Cells.

CD8 T cell-deficient, MHC class I-null (KbDb KO) animals received 3 Gy TBI on Day −1 and 2 mg anti-CD154 on Day 0, followed by i.v. injection of 9×10⁶ adoptively transferred CD8 T cells from either wild-type B6 animals (WT) or PD-1 KO animals and 25×10⁶ T cell-depleted B10.A (fully MHC-mismatched) BMCs. (FIG. 6A) shows the percent donor chimerism in the B220+ lineage (representative of all lineages) of the peripheral blood for each individual animal (FIG. 6B) shows the average and standard deviation of peripheral B220 chimerism for both groups. (FIG. 6C) shows the incidence of chimerism at 7 weeks post-BMT.

### DEFINITIONS

As used herein, a “biologically active portion” of a polypeptide includes a fragment of a polypeptide of interest which participates in an interaction, e.g., an intramolecular or an inter-molecular interaction, e.g., a binding or catalytic interaction. Biologically active portions include peptides comprising amino acid sequences sufficiently homologous to or derived from the amino acid sequence of the protein which include fewer amino acids than the full length, natural protein, and exhibit at least one activity of the natural protein. Biological active portions/functional domains can be identified by a variety of techniques including truncation analysis, site-directed mutagenesis, and proteolysis. Mutants or proteolytic fragments can be assayed for activity by an appropriate biochemical or biological (e.g., genetic) assay. In some embodiments, a functional domain is independently folded. Typically, biologically active portions comprise a domain or motif with at least one activity of a protein, e.g., an extracellular domain required for binding to a ligand. A biologically active portion/functional domain of a protein can be a polypeptide which is, for example, 10, 25, 50, 100, 200 or more amino acids in length.

“Graft”, as used herein, refers to a body part, organ, tissue, or cells. Organs such as liver, kidney, heart or lung, or other body parts, such as bone or skeletal matrix, tissue, such as skin, intestines, endocrine glands, or progenitor stem cells of various types, are all examples of grafts.

“Hematopoietic stem cell”, as used herein, refers to a cell, e.g., a bone marrow cell, or a fetal liver or spleen cell, which is capable of developing into all myeloid and lymphoid lineages and by virtue of being able to self-renew can provide long term hematopoietic reconstitution. Purified preparations of hematopoietic cells or preparations, such as bone marrow, which include other cell types, can be used in methods of the invention. The preparation should include immature cells, i.e., undifferentiated hematopoietic stem cells; these desired cells can be separated out of a preparation or a complex preparation can be administered. E.g., in the case of bone marrow stem cells, the desired primitive cells can be separated out of a preparation or a complex bone marrow sample including such cells can be used. Hematopoietic stem cells can be from fetal, neonatal, immature or mature animals. Stem cells derived from the cord blood of the recipient or the donor can be used in methods described herein. See U.S. Pat. No. 5,192,553, hereby incorporated by reference, and U.S. Pat. No. 5,004,681, hereby incorporated by reference. In some embodiments, donor peripheral blood hematopoietic stem cells are used.

An “inhibitor of a costimulatory pathway” as used herein, refers to an agent which binds a member of a costimulatory ligand/counter-ligand pair and inhibits the interaction between the ligand and counter-ligand or which disrupts the ability of the bound member to transduce a signal. In some embodiments, the inhibitor is an antibody to the ligand or counter ligand, a soluble ligand (soluble fragment of the counter ligand), a soluble counter ligand (soluble fragment of the counter ligand), or other protein, peptide or other molecule (e.g., a small molecule) which binds specifically to the counter-ligand or ligand, e.g., a protein or peptide selected by virtue of its ability to bind the ligand or counter ligand in an affinity assay. In some embodiments, an inhibitor of a costimulatory pathway inhibits a CD40-CD154 pathway, or a CD28-B7 pathway. As used herein, the term “antibody” includes portions of antibodies that retain the ability to specifically bind antigen.

“PD-1”: As used herein, the term “PD-1”, also known as Programmed Death 1, Programmed Cell Death 1, PDCD1, and CD279, refers to a PD-1 polypeptide. PD-1 is a member of the B7 superfamily and a homolog of CD28. PD-1 has a single extracellular Ig-like variable domain and an immunoreceptor tyrosine-based inhibitory motif (ITIM) in its cytoplasmic domain. A nucleotide sequence encoding a human PD-1 polypeptide is found in GenBank under Acc. No. NM_{—}005018.2. An exemplary human PD-1 polypeptide sequence is found under Acc. No. NP_{—}005009.2. “PD-1”, as used herein, includes human and non-human forms of PD-1. Sequences of non-human PD-1 genes and polypeptides are known. For example, murine and rat PD-1 polypeptide sequences are found under Acc. Nos. NP_{—}032824.1 and XP_{—}237422.3, respectively. The GenBank database sequence entries above are incorporated herein by reference.

An amino acid sequence of a human PD-1 polypeptide, found under GenBank Acc. No. NP_{—}005009.2, is as follows:

(SEQ ID NO: 1)
MQIPQAPWPVVWAVLQLGWRPGWFLDSPDRPWNPPTFSPALLVVTEGDN
ATFTCSFSNTSESFVLNWYRMSPSNQTDKLAAFPEDRSQPGQDCRFRVT
QLPNGRDFHMSVVRARRNDSGTYLCGAISLAPKAQIKESLRAELRVTER
RAEVPTAHPSPSPRPAGQFQTLVVGVVGGLLGSLVLLVWVLAVICSRAA
RGTIGARRTGQPLKEDPSAVPVFSVDYGELDFQWREKTPEPPVPCVPEQ
TEYATIVFPSGMGTSSPARRGSADGPRSAQPLRPEDGHCSWPL

A nucleotide sequence encoding a human PD-1 polypeptide, found in GenBank under Acc. No. NM_{—}005018.2, is as follows:

(SEQ ID NO: 2)
AGTTTCCCTTCCGCTCACCTCCGCCTGAGCAGTGGAGAAGGCGGCACTCTGGTGGGGCTGCTCCAGGCAT
GCAGATCCCACAGGCGCCCTGGCCAGTCGTCTGGGCGGTGCTACAACTGGGCTGGCGGCCAGGATGGTTC
TTAGACTCCCCAGACAGGCCCTGGAACCCCCCCACCTTCTCCCCAGCCCTGCTCGTGGTGACCGAAGGGG
ACAACGCCACCTTCACCTGCAGCTTCTCCAACACATCGGAGAGCTTCGTGCTAAACTGGTACCGCATGAG
CCCCAGCAACCAGACGGACAAGCTGGCCGCCTTCCCCGAGGACCGCAGCCAGCCCGGCCAGGACTGCCGC
TTCCGTGTCACACAACTGCCCAACGGGCGTGACTTCCACATGAGCGTGGTCAGGGCCCGGCGCAATGACA
GCGGCACCTACCTCTGTGGGGCCATCTCCCTGGCCCCCAAGGCGCAGATCAAAGAGAGCCTGCGGGCAGA
GCTCAGGGTGACAGAGAGAAGGGCAGAAGTGCCCACAGCCCACCCCAGCCCCTCACCCAGGCCAGCCGGC
CAGTTCCAAACCCTGGTGGTTGGTGTCGTGGGCGGCCTGCTGGGCAGCCTGGTGCTGCTAGTCTGGGTCC
TGGCCGTCATCTGCTCCCGGGCCGCACGAGGGACAATAGGAGCCAGGCGCACCGGCCAGCCCCTGAAGGA
GGACCCCTCAGCCGTGCCTGTGTTCTCTGTGGACTATGGGGAGCTGGATTTCCAGTGGCGAGAGAAGACC
CCGGAGCCCCCCGTGCCCTGTGTCCCTGAGCAGACGGAGTATGCCACCATTGTCTTTCCTAGCGGAATGG
GCACCTCATCCCCCGCCCGCAGGGGCTCAGCTGACGGCCCTCGGAGTGCCCAGCCACTGAGGCCTGAGGA
TGGACACTGCTCTTGGCCCCTCTGACCGGCTTCCTTGGCCACCAGTGTTCTGCAGACCCTCCACCATGAG
CCCGGGTCAGCGCATTTCCTCAGGAGAAGCAGGCAGGGTGCAGGCCATTGCAGGCCGTCCAGGGGCTGAG
CTGCCTGGGGGCGACCGGGGCTCCAGCCTGCACCTGCACCAGGCACAGCCCCACCACAGGACTCATGTCT
CAATGCCCACAGTGAGCCCAGGCAGCAGGTGTCACCGTCCCCTACAGGGAGGGCCAGATGCAGTCACTGC
TTCAGGTCCTGCCAGCACAGAGCTGCCTGCGTCCAGCTCCCTGAATCTCTGCTGCTGCTGCTGCTGCTGC
TGCTGCTGCCTGCGGCCCGGGGCTGAAGGCGCCGTGGCCCTGCCTGACGCCCCGGAGCCTCCTGCCTGAA
CTTGGGGGCTGGTTGGAGATGGCCTTGGAGCAGCCAAGGTGCCCCTGGCAGTGGCATCCCGAAACGCCCT
GGACGCAGGGCCCAAGACTGGGCACAGGAGTGGGAGGTACATGGGGCTGGGGACTCCCCAGGAGTTATCT
GCTCCCTGCAGGCCTAGAGAAGTTTCAGGGAAGGTCAGAAGAGCTCCTGGCTGTGGTGGGCAGGGCAGGA
AACCCCTCCACCTTTACACATGCCCAGGCAGCACCTCAGGCCCTTTGTGGGGCAGGGAAGCTGAGGCAGT
AAGCGGGCAGGCAGAGCTGGAGGCCTTTCAGGCCCAGCCAGCACTCTGGCCTCCTGCCGCCGCATTCCAC
CCCAGCCCCTCACACCACTCGGGAGAGGGACATCCTACGGTCCCAAGGTCAGGAGGGCAGGGCTGGGGTT
GACTCAGGCCCCTCCCAGCTGTGGCCACCTGGGTGTTGGGAGGGCAGAAGTGCAGGCACCTAGGGCCCCC
CATGTGCCCACCCTGGGAGCTCTCCTTGGAACCCATTCCTGAAATTATTTAAAGGGGTTGGCCGGGCTCC
CACCAGGGCCTGGGTGGGAAGGTACAGGCGTTCCCCCGGGGCCTAGTACCCCCGCCGTGGCCTATCCACT
CCTCACATCCACACACTGCACCCCCACTCCTGGGGCAGGGCCACCAGCATCCAGGCGGCCAGCAGGCACC
TGAGTGGCTGGGACAAGGGATCCCCCTTCCCTGTGGTTCTATTATATTATAATTATAATTAAATATGAGA
GCATGCTAAGGAAAA

“PD-1 agonist”: As used herein, the term “PD-1 agonist” is an agent that agonizes PD-1 and/or which induces or increases one or more PD-1 associated activities. This includes, by way of example PD-1 agonistic antibodies, soluble ligands of PD-1, such as soluble PD-L1 and soluble PD-L2, and fragments and derivatives thereof such as oligomeric (e.g., bivalent, trimeric) forms, fusion proteins, and variants thereof produced by recombinant or protein synthesis. In addition, PD-1 agonists include small molecules, and aptamers which comprise RNA or DNA molecules that can be substituted for antibodies. In some embodiments, a PD-1 agonist is PD-L1-Ig. In some embodiments, a PD-1 agonist is PD-L2-Ig. PD-1 associated activities which may be induced or increased by a PD-1 agonist include inhibition of T cell proliferation, inhibition of T cell cytokine (e.g., IL-2, IFN-γ, IL-10) secretion (see Freeman et al., J. Exp Med. 2000 Oct. 2; 192(7): 1027-1034; and Latchman et al., Nat Immunol. 2001 March; 2(3):261-8).

“PD-L1”: As used herein, the term “PD-L1”, also known as Programmed Death 1 Ligand 1, Programmed Cell Death 1 Ligand 1, PDCD1L1, B7H1, and CD274, refers to a PD-L1 polypeptide. PD-L1 is a homolog of B7 and is a ligand for PD-1. A nucleotide sequence encoding a human PD-L1 polypeptide is found in GenBank under Acc. No. NM_{—}014143.2. An exemplary human PD-L1 polypeptide sequence is found under Acc. No. NP_{—}054862.1. “PD-L1”, as used herein, includes human and non-human forms of PD-L1. Sequences of non-human PD-L1 genes and polypeptides are known. For example, murine and rat PD-L1 polypeptide sequences are found under Acc. Nos. NP_{—}068693.1 and XP_{—}574652.2, respectively. The GenBank database sequence entries above are incorporated herein by reference.

An amino acid sequence of a human PD-L1 polypeptide, found under GenBank Acc. No. NP_{—}054862.1, is as follows:

(SEQ ID NO: 3)
MRIFAVFIFMTYWHLLNAFTVTVPKDLYVVEYGSNMTIECKFPVEKQLD
LAALIVYWEMEDKNIIQFVHGEEDLKVQHSSYRQRARLLKDQLSLGNAA
LQITDVKLQDAGVYRCMISYGGADYKRITVKVNAPYNKINQRILVVDPV
TSEHELTCQAEGYPKAEVIWTSSDHQVLSGKTTTTNSKREEKLFNVTST
LRINTTTNEIFYCTFRRLDPEENHTAELVIPELPLAHPPNERTHLVILG
AILLCLGVALTFIFRLRKGRMMDVKKCGIQDTNSKKQSDTHLEET

The signal peptide of PD-L1 is located at residues 1-18 of SEQ ID NO:3. The extracellular domain includes residues 1-238 of SEQ ID NO:3 (or residues 19-238, following cleavage of the signal peptide).

A nucleotide sequence encoding a human PD-L1 polypeptide, found in GenBank under Acc. No. NM_{—}014143.2, is as follows:

(SEQ ID NO: 4)
CGAGGCTCCGCACCAGCCGCGCTTCTGTCCGCCTGCAGGGCATTCCAGAAAGATGAGGATATTTGCTGTC
TTTATATTCATGACCTACTGGCATTTGCTGAACGCATTTACTGTCACGGTTCCCAAGGACCTATATGTGG
TAGAGTATGGTAGCAATATGACAATTGAATGCAAATTCCCAGTAGAAAAACAATTAGACCTGGCTGCACT
AATTGTCTATTGGGAAATGGAGGATAAGAACATTATTCAATTTGTGCATGGAGAGGAAGACCTGAAGGTT
CAGCATAGTAGCTACAGACAGAGGGCCCGGCTGTTGAAGGACCAGCTCTCCCTGGGAAATGCTGCACTTC
AGATCACAGATGTGAAATTGCAGGATGCAGGGGTGTACCGCTGCATGATCAGCTATGGTGGTGCCGACTA
CAAGCGAATTACTGTGAAAGTCAATGCCCCATACAACAAAATCAACCAAAGAATTTTGGTTGTGGATCCA
GTCACCTCTGAACATGAACTGACATGTCAGGCTGAGGGCTACCCCAAGGCCGAAGTCATCTGGACAAGCA
GTGACCATCAAGTCCTGAGTGGTAAGACCACCACCACCAATTCCAAGAGAGAGGAGAAGCTTTTCAATGT
GACCAGCACACTGAGAATCAACACAACAACTAATGAGATTTTCTACTGCACTTTTAGGAGATTAGATCCT
GAGGAAAACCATACAGCTGAATTGGTCATCCCAGAACTACCTCTGGCACATCCTCCAAATGAAAGGACTC
ACTTGGTAATTCTGGGAGCCATCTTATTATGCCTTGGTGTAGCACTGACATTCATCTTCCGTTTAAGAAA
AGGGAGAATGATGGATGTGAAAAAATGTGGCATCCAAGATACAAACTCAAAGAAGCAAAGTGATACACAT
TTGGAGGAGACGTAATCCAGCATTGGAACTTCTGATCTTCAAGCAGGGATTCTCAACCTGTGGTTTAGGG
GTTCATCGGGGCTGAGCGTGACAAGAGGAAGGAATGGGCCCGTGGGATGCAGGCAATGTGGGACTTAAAA
GGCCCAAGCACTGAAAATGGAACCTGGCGAAAGCAGAGGAGGAGAATGAAGAAAGATGGAGTCAAACAGG
GAGCCTGGAGGGAGACCTTGATACTTTCAAATGCCTGAGGGGCTCATCGACGCCTGTGACAGGGAGAAAG
GATACTTCTGAACAAGGAGCCTCCAAGCAAATCATCCATTGCTCATCCTAGGAAGACGGGTTGAGAATCC
CTAATTTGAGGGTCAGTTCCTGCAGAAGTGCCCTTTGCCTCCACTCAATGCCTCAATTTGTTTTCTGCAT
GACTGAGAGTCTCAGTGTTGGAACGGGACAGTATTTATGTATGAGTTTTTCCTATTTATTTTGAGTCTGT
GAGGTCTTCTTGTCATGTGAGTGTGGTTGTGAATGATTTCTTTTGAAGATATATTGTAGTAGATGTTACA
ATTTTGTCGCCAAACTAAACTTGCTGCTTAATGATTTGCTCACATCTAGTAAAACATGGAGTATTTGTAA
AAAAAAAAAAAAA

“PD-L2”: As used herein, the term “PD-L2”, also known as Programmed Death 1 Ligand 2, Programmed Cell Death 1 Ligand 2, B7-DC, PDCD1L2, and CD273, refers to a PD-L2 polypeptide. PD-L2 is a B7 homolog and is a ligand for PD-1. A nucleotide sequence encoding a human PD-L2 polypeptide is found in GenBank under Acc. No. NM_{—}025239.3. An exemplary human PD-L2 polypeptide sequence is found under Acc. No. NP_{—}079515.1. “PD-L2”, as used herein, includes human and non-human forms of PD-L2. Sequences of non-human PD-L2 genes and polypeptides are known. For example, murine and rat PD-L2 polypeptide sequences are found under Acc. Nos. NP_{—}067371.1 and XP_{—}219777.4, respectively. The GenBank database sequence entries above are incorporated herein by reference.

An amino acid sequence of a human PD-L2 polypeptide, found under GenBank Acc. No. NP_{—}079515.1, is as follows:

(SEQ ID NO: 5)
MIFLLLMLSLELQLHQIAALFTVTVPKELYIIEHGSNVTLECNFDTGSH
VNLGAITASLQKVENDTSPHRERATLLEEQLPLGKASFHIPQVQVRDEG
QYQCIIIYGVAWDYKYLTLKVKASYRKINTHILKVPETDEVELTCQATG
YPLAEVSWPNVSVPANTSHSRTPEGLYQVTSVLRLKPPPGRNFSCVFWN
THVRELTLASIDLQSQMEPRTHPTWLLHIFIPSCIIAFIFIATVIALRK
QLCQKLYSSKDTTKRPVTTTKREVNSAI

A nucleotide sequence encoding a human PD-L2 polypeptide, found in GenBank under Acc. No. NM_{—}025239.3, is as follows:

(SEQ ID NO: 6)
GCAAACCTTAAGCTGAATGAACAACTTTTCTTCTCTTGAATATATCTTAACGCCAAATTTTGAGTGCTTT
TTTGTTACCCATCCTCATATGTCCCAGCTAGAAAGAATCCTGGGTTGGAGCTACTGCATGTTGATTGTTT
TGTTTTTCCTTTTGGCTGTTCATTTTGGTGGCTACTATAAGGAAATCTAACACAAACAGCAACTGTTTTT
TGTTGTTTACTTTTGCATCTTTACTTGTGGAGCTGTGGCAAGTCCTCATATCAAATACAGAACATGATCT
TCCTCCTGCTAATGTTGAGCCTGGAATTGCAGCTTCACCAGATAGCAGCTTTATTCACAGTGACAGTCCC
TAAGGAACTGTACATAATAGAGCATGGCAGCAATGTGACCCTGGAATGCAACTTTGACACTGGAAGTCAT
GTGAACCTTGGAGCAATAACAGCCAGTTTGCAAAAGGTGGAAAATGATACATCCCCACACCGTGAAAGAG
CCACTTTGCTGGAGGAGCAGCTGCCCCTAGGGAAGGCCTCGTTCCACATACCTCAAGTCCAAGTGAGGGA
CGAAGGACAGTACCAATGCATAATCATCTATGGGGTCGCCTGGGACTACAAGTACCTGACTCTGAAAGTC
AAAGCTTCCTACAGGAAAATAAACACTCACATCCTAAAGGTTCCAGAAACAGATGAGGTAGAGCTCACCT
GCCAGGCTACAGGTTATCCTCTGGCAGAAGTATCCTGGCCAAACGTCAGCGTTCCTGCCAACACCAGCCA
CTCCAGGACCCCTGAAGGCCTCTACCAGGTCACCAGTGTTCTGCGCCTAAAGCCACCCCCTGGCAGAAAC
TTCAGCTGTGTGTTCTGGAATACTCACGTGAGGGAACTTACTTTGGCCAGCATTGACCTTCAAAGTCAGA
TGGAACCCAGGACCCATCCAACTTGGCTGCTTCACATTTTCATCCCCTTCTGCATCATTGCTTTCATTTT
CATAGCCACAGTGATAGCCCTAAGAAAACAACTCTGTCAAAAGCTGTATTCTTCAAAAGACACAACAAAA
AGACCTGTCACCACAACAAAGAGGGAAGTGAACAGTGCTATCTGAACCTGTGGTCTTGGGAGCCAGGGTG
ACCTGATATGACATCTAAAGAAGCTTCTGGACTCTGAACAAGAATTCGGTGGCCTGCAGAGCTTGCCATT
TGCACTTTTCAAATGCCTTTGGATGACCCAGCACTTTAATCTGAAACCTGCAACAAGACTAGCCAACACC
TGGCCATGAAACTTGCCCCTTCACTGATCTGGACTCACCTCTGGAGCCTATGGCTTTAAGCAAGCACTAC
TGCACTTTACAGAATTACCCCACTGGATCCTGGACCCACAGAATTCCTTCAGGATCCTTCTTGCTGCCAG
ACTGAAAGCAAAAGGAATTATTTCCCCTCAAGTTTTCTAAGTGATTTCCAAAAGCAGAGGTGTGTGGAAA
TTTCCAGTAACAGAAACAGATGGGTTGCCAATAGAGTTATTTTTTATCTATAGCTTCCTCTGGGTACTAG
AAGAGGCTATTGAGACTATGAGCTCACAGACAGGGCTTCGCACAAACTCAAATCATAATTGACATGTTTT
ATGGATTACTGGAATCTTGATAGCATAATGAAGTTGTTCTAATTAACAGAGAGCATTTAAATATACACTA
AGTGCACAAATTGTGGAGTAAAGTCATCAAGCTCTGTTTTTGAGGTCTAAGTCACAAAGCATTTGTTTTA
ACCTGTAATGGCACCATGTTTAATGGTGGTTTTTTTTTTGAACTACATCTTTCCTTTAAAAATTATTGGT
TTCTTTTTATTTGTTTTTACCTTAGAAATCAATTATATACAGTCAAAAATATTTGATATGCTCATACGTT
GTATCTGCAGCAATTTCAGATAAGTAGCTAAAATGGCCAAAGCCCCAAACTAAGCCTCCTTTTCTGGCCC
TCAATATGACTTTAAATTTGACTTTTCAGTGCCTCAGTTTGCACATCTGTAATACAGCAATGCTAAGTAG
TCAAGGCCTTTGATAATTGGCACTATGGAAATCCTGCAAGATCCCACTACATATGTGTGGAGCAGAAGGG
TAACTCGGCTACAGTAACAGCTTAATTTTGTTAAATTTGTTCTTTATACTGGAGCCATGAAGCTCAGAGC
ATTAGCTGACCCTTGAACTATTCAAATGGGCACATTAGCTAGTATAACAGACTTACATAGGTGGGCCTAA
AGCAAGCTCCTTAACTGAGCAAAATTTGGGGCTTATGAGAATGAAAGGGTGTGAAATTGACTAACAGACA
AATCATACATCTCAGTTTCTCAATTCTCATGTAAATCAGAGAATGCCTTTAAAGAATAAAACTCAATTGT
TATTCTTCAACGTTCTTTATATATTCTACTTTTGGGTA

“Promoting acceptance of a graft” as used herein, refers to any of increasing the time a graft is accepted or is functional or decreasing a graft recipient's immune response to the graft, e.g., by the induction of tolerance.

“Tolerance”, as used herein, refers to an inhibition of a graft recipient's immune response which would otherwise occur, e.g., in response to the introduction of a nonself MHC antigen or other allogeneic or xenogeneic antigen into the recipient. Tolerance can involve humoral, cellular, or both humoral and cellular responses. Tolerance, as used herein, refers not only to complete immunologic tolerance to an antigen, but to partial immunologic tolerance, i.e., a degree of tolerance to an antigen which is greater than what would be seen if a method of the invention were not employed. Tolerance, as used herein, refers to a donor antigen-specific inhibition of the immune system as opposed to the broad spectrum inhibition of the immune system seen with immunosuppressants.

### DETAILED DESCRIPTION OF CERTAIN PREFERRED EMBODIMENTS OF THE INVENTION

A major goal of organ transplantation is to avoid chronic rejection and complications associated with long-term immunosuppressive therapy. Methods described herein combine hematopoietic cell transplantation and PD-1 agonism to induce tolerance and promote graft acceptance. These methods arise from the discovery that PD-1 regulates CD8 T cell tolerance, and that PD-1 ligation can overcome CD8 T cell resistance. The combination of PD-1 agonism and hematopoietic cell transplantation provide a powerful means of promoting tolerance.

The Induction of Tolerance with Bone Marrow Transplantation and PD-1 Agonism

The following procedures are designed to promote acceptance of a transplanted graft, and lengthen the time the graft survives in a recipient (e.g., an allogeneic or xenogeneic recipient) prior to rejection. The graft can be any organ, tissue, or cells, e.g., a liver, kidney, pancreas, heart, lung, or skin graft.

Methods of inducing tolerance and promoting graft acceptance include transplantation of tolerance-inducing cells, e.g., bone marrow cells, e.g., hematopoietic stem cells; and administration of a PD-1 agonist.

Cells for Stem Cell Transplantation

Sources of hematopoietic stem cells include bone marrow cells, mobilized peripheral blood cells, and cord blood cells. In some embodiments, mobilized peripheral stem cells are used in methods described herein. In vitro expanded hematopoietic cells can be used (see, e.g., Petzer et al., 1996, Proc. Natl. Acad. Sci. USA 93:1470; Zundstra et al., 1994, BioTechnology 12:909; and WO 95 11692).

Bone marrow cells (BMC), or another source of hematopoietic stem cells, e.g., donor peripheral hematopoietic stem cells, of the donor are injected into the recipient. Donor hematopoietic stem cells home to appropriate sites of the recipient and grow contiguously with remaining host cells and proliferate, forming a chimeric lymphohematopoietic population. By this process, newly forming and pre-existing B cells (and the antibodies they produce) are exposed to donor antigens, so that a transplant will be recognized as self. Tolerance to the donor is also observed at the T cell level in animals in which hematopoietic stem cell, e.g., BMC, engraftment has been achieved. When an organ graft is placed in such a recipient several months after bone marrow mixed chimerism has been induced the graft should be accepted by both the humoral and the cellular aims of the immune system. This approach permits organ transplantation to be performed after transplant of hematopoietic cells, e.g., BMT, e.g., a fetal liver suspension, when normal health and immunocompetence will have been restored at the time of organ transplantation. In some embodiments, hematopoietic cells and a graft are implanted at about the same time (e.g., within one week, five days, three days, or on the same day). Introduction of hematopoietic cells and a PD-1 agonist can also be practiced after organ transplantation. In these embodiments, the recipient would have been receiving conventional immunosuppression to block rejection of the graft. After administration of the tolerogenic hematopoietic cells and a PD-1 agonist, and induction of tolerance, immunosuppression would be withdrawn.

PD-1 Agonists

PD-1 is a receptor expressed on activated T and B lymphocytes. PD-1 agonists induce or increase one or more PD-1 associated activities. In some embodiments, a PD-1 agonist inhibits one or more of T cell intracellular signaling, proliferation, and cytokine production. Natural ligands for PD-1 include PD-L1 and PD-L2. These PD-1 ligands may be employed as PD-1 agonists in the methods described herein. Other types of PD-1 agonists include PD-1 agonistic antibodies, small molecules, and aptamers which comprise RNA or DNA molecules that can be substituted for antibodies. In some embodiments, a PD-1 agonist is a soluble form of a PD-1 ligand (e.g., soluble PD-L1, soluble PD-L2). Soluble forms of PD-1 ligands typically include the extracellular domain of the ligand, or a portion thereof sufficient to bind to, and agonize, PD-1. In some embodiments, a soluble portion of PD-L1 includes amino acids 19-238 (or amino acids ˜20-237, or a portion within including about 100, 150, 170 180, 190 amino acid residues) of SEQ ID NO:3. A soluble PD-L2 can include an analogous portion. In some embodiments, a PD-1 agonist is a soluble PD-1 ligand fused to a heterologous polypeptide (e.g., a heterologous polypeptide that increases the circulating half-life of the ligand, such as an Fc region of an immunoglobulin). In some embodiments, a PD-1 ligand is fused to an Fc portion of a human IgG1. PD-1 ligand-Fc fusions are referred to herein “PD-L1-Ig” and “PD-L2-Ig”. PD-L1-Ig and PD-L2-Ig are described, e.g., in Freeman et al., J. Exp Med. 2000 Oct. 2; 192(7): 1027-1034; Latchman et al., Nat Immunol. 2001 March; 2(3):261-8; Watson et al., Invest Ophthalmol Vis Sci. 2006 August; 47(8):3417-22; and Youngnak et al., Biochem Biophys Res Comm. 2003; 307:672-677.

A PD-1 agonist can be administered to a subject prior to, simultaneously with, and/or after administration of hematopoietic stem cells. In some embodiments, a PD-1 agonist is administered immediately prior to, and after, administration of hematopoietic cells (e.g., at day 0, and daily for 7-14 days post transplant). A PD-1 agonist can be administered prior to, simultaneously with, and/or after administration of a graft (e.g., an organ graft, e.g., a heart, kidney, or liver graft). In some embodiments, a PD-1 agonist is administered both at the time of, and after, hematopoietic cell transplantation, and at the time of, and after, organ graft transplantation. In some embodiments, a PD-1 agonist is administered at the time of, and after, hematopoietic cell transplantation, and is not administered at the time of organ graft transplantation.

In some embodiments, hematopoietic cells are treated with a PD-1 agonist ex vivo, prior to administration of the cells to a recipient.

Conditioning Regimens and Additional Agents

The recipient can be exposed to whole body irradiation or chemotherapy to create hematopoietic space for engraftment of donor hematopoietic cells, e.g., 300 cGy of whole body X-rays. Chemotherapeutics suitable for creating hematopoietic space include, e.g., cyclophosphamide, busulfan, fludararabine, or any combination thereof. The chemotherapeutic agent can be a radiomimetic (i.e. an agent that interacts with DNA in a mechanism similar to ionizing radiation) or a non-radiomimetic. In one embodiment, a single-agent chemotherapeutic regimen is administered. For example, cyclophosphamide can be administered (preferably, intravenously) at a dose of 50-60 mg/kg/day for three consecutive days (e.g., on days −5, −4 and −3 prior to administration of stem cells or bone marrow on day 0). Alternatively, a combination of agents, e.g., cyclophosphamide, busulfan and/or fludararabine, can be used. Exemplary combinations include cyclophosphamide and fludararabine, and cyclophosphamide and busulfan. Analogues of these compounds may also be incorporated into the final regimen, e.g., substituting treosulfan for busulfan; purine or nucleoside analogs of these agents, e.g., fludarabine. The combination of agents can be administered sequentially or concurrently.

In some embodiments, the methods include a treatment that creates thymic space, e.g., by administering to the recipient, an amount of thymic irradiation sufficient to kill or otherwise inactivate recipient thymocytes. By way of example, 700 cGy of thymic irradiation can be administered to the recipient.

The methods can also include administering to the recipient, one or more doses of at least one antibody which depletes T cells. The antibodies can be administered before, on, or after, the day of bone marrow transplantation, and/or before, on, or after the day of graft implantation. Suitable antibodies include, polyclonal anti-thymocyte globulin (ATG), anti-CD4, anti-CD8, and anti-CD2 antibodies. In some embodiments, a method includes administering to the recipient, an anti-CD2 monoclonal antibody, e.g., administering to the recipient, at least one dose, and possibly two, three or four, doses of an anti-CD2 monoclonal antibody, e.g., before or up to the day of hematopoietic cell (or graft) implantation.

In some embodiments, an anti-CD2 antibody used in a method described herein is a monoclonal antibody which binds an epitope which overlaps, or which is similar to, the epitope bound by rat monoclonal anti-CD2 antibody BTI-322 or the humanized anti-CD2 antibody, MEDI-507. For example, a preferred anti-CD2 antibody is one which can inhibit the binding of BTI-322 to its epitope, inhibit the binding of MEDI-507 to its epitope, be inhibited in binding to its epitope by BTI-322, or be inhibited in binding to its epitope by MEDI-507. BTI-322 is described in U.S. Pat. No. 5,817,311, hereby incorporated by reference. BTI-322 has been deposited with the ATCC as accession number HB 11423. MEDI-507 is described in PCT/US97/12645 (WO9903502, published Jan. 28, 1999), hereby incorporated by reference. MEDI-507 is a humanized form of a rat IgG2b kappa monoclonal antibody raised against human lymphocytes and reactive with the CD2 antigen on the lymphocytes. MEDI-507 can be supplied as a solution in phosphate buffered saline (pH 7.4) in vials containing 15 mg for intravenous use.

By way of example, three or four doses of an anti-CD2 monoclonal antibody, e.g., MEDI-507, can be administered. In some embodiments, doses of 0.1 mg/kg-1.0 mg/kg of the anti-CD2 monoclonal antibody MEDI-507 are used. Further examples of modifications of hematopoietic cell grafting methods are described in U.S. Pat. No. 6,877,514; U.S. Pat. No. 6,280,957, and U.S. Pat. Pub. No. 20080175850, the contents of which are incorporated by reference herein.

In the methods of inducing T cell tolerance and promoting graft acceptance described herein, T cells, particularly, thymic or lymph node T cells, can be further suppressed by administering to the recipient an immunosuppressive agent, e.g., a non-chronic course of an immunosuppressive agent such as cyclosporine, FK506, or rapamycin. The immunosuppressant is administered in an amount sufficient such that T cell dependent rejection is inhibited. In some embodiments, administration does not extend beyond 2, 4, 6, 12, or 18 months after graft implantation. By way of example, starting on the day after transplantation (day 1), the dose of cyclosporine can be 4 mg/kg given twice a day, and adjusted to provide a trough whole blood concentration of 250-500 ng/mL, as measured by a monoclonal antibody based assay (or the equivalent if a different assay or serum rather than whole blood are used), then tapered and discontinued over a period of several months. In some embodiments, use of a PD-1 agonist in conjunction with hematopoietic cell transplantation induces tolerance such that graft rejection is delayed or reduced in a subject receiving a dose of immunosuppressive therapy that, on its own, would normally be ineffective to prevent rejection.

In some embodiments, the methods include administration of a treatment that depletes B cells, e.g., an anti-B cell antibody, such as rituximab, e.g., before, during, or after hematopoietic stem cell and/or graft implantation.

The methods can also include administration of inhibitors of a costimulatory pathway, e.g., inhibitors of a CD154-CD40 interaction, or inhibitors of a CD28-B7 interaction. In some embodiments, an anti-CD154 (e.g., ABI793, Novartis Pharma AG, Basel, Switzerland) or anti-CD40 monoclonal antibody is used. In some embodiments, a soluble CTLA4, e.g., a CTLA4-IgG fusion protein, is used as an inhibitor of a costimulatory pathway (see, e.g., U.S. Pat. No. 5,851,795).

In the methods described herein, it may also be necessary or desirable to splenectomize the recipient.

The methods of introducing hematopoietic stem cells and a PD-1 agonist can promote acceptance of the hematopoietic stem cell graft. In addition, the cells and agonist treatment can promote acceptance of a separate graft (e.g., a solid organ graft, e.g., a heart, liver, or kidney graft). The donor of the hematopoietic stem cells and graft may be the same individual, or different individuals. In some embodiments, a donor is related to the recipient (e.g., the donor is a sibling, parent or cousin). In other embodiments, the donor is unrelated. The donor and recipient may be mismatched for MHC alleles, or may be matched. Grafts may be from a living donor, or a cadaver.

Xenogeneic Embodiments

The methods for inducing tolerance and promoting graft acceptance as described herein are applicable to allogeneic and xenogeneic (cross-species) transplantation. Preparation of a xenogeneic recipient for transplantation, and maintenance of the recipient after transplantation, can include any of the steps described above, and, additionally can include additional use of steps and agents described below. In some embodiments in which xenogeneic transplantation is practiced, a donor mammal is a swine (e.g., a miniature swine, e.g., a wholly or partially inbred miniature swine) and the recipient is a primate.

Natural antibodies present in humans and Old World primates react with galactosyl-α-1,3-galactose moieties present on swine tissues. In some embodiments, natural antibodies in a recipient of xenogeneic tissue are eliminated by organ perfusion. Also, or alternatively, the cells, tissues, or organs used for transplantation may be genetically modified such that they are not recognized by natural antibodies of the host (e.g., swine cells used for administration to a primate are α-1,3-galactosyltransferase deficient).

In some embodiments, a recipient is thymectomized, or receives thymic irradiation.

The recipient can be treated with an agent that depletes complement, such as cobra venom factor (e.g., at approx. 5-10 mg/d, at days −1).

In some embodiments, maintenance therapy of a xenogeneic graft recipient (e.g., beginning immediately prior to, and continuing for at least a few days after transplantation) includes treatment with a human anti-human CD154 mAb (e.g., ABI793, Novartis Pharma AG, Basel, Switzerland; ˜25 mg/kg). Mycophenolate mofetil (MMF; 25-110 mg/kd/d) may be administered to maintain whole blood levels to a desirable level. Methylprednisolone may also be administered, e.g., beginning on the day of transplantation, tapering thereafter over the next 3-4 weeks.

Various agents useful for supportive therapy (e.g., at days 0-14) include anti-inflammatory agents such as prostacyclin, dopamine, ganiclovir, levofloxacin, cimetidine, heparin, antithrombin, erythropoietin, and aspirin.

In some embodiments, donor stromal tissue is administered. In some embodiments, donor stromal tissue it is obtained from fetal or juvenile liver, thymus, and/or spleen, may be implanted into the recipient, preferably in the kidney capsule. Thymic tissue can be prepared for transplantation by implantation under the autologous kidney capsule for revascularization.

Stem cell engraftment and hematopoiesis across disparate species barriers can be enhanced by providing a hematopoietic stromal environment from the donor species. The stromal matrix supplies species-specific factors that are required for interactions between hematopoietic cells and their stromal environment, such as hematopoietic growth factors, adhesion molecules, and their ligands.

As liver is the major site of hematopoiesis in the fetus, fetal liver can also serve as an alternative to bone marrow as a source of hematopoietic stem cells. The thymus is the major site of T cell maturation. Each organ includes an organ specific stromal matrix that can support differentiation of the respective undifferentiated stem cells implanted into the host. As an added precaution against GVHD, thymic stromal tissue can be irradiated prior to transplantation, e.g., irradiated at 1000 rads. As an alternative or an adjunct to implantation, fetal liver cells can be administered in fluid suspension.

The use of xenogeneic donors allows the possibility of using bone marrow cells and organs from the same animal, or from genetically matched animals.

The approaches described above are designed to synergistically prevent the problem of transplant rejection.

The methods of the invention may be employed in combination, as described, or in part.

The methods described herein may be combined with methods described in U.S. Pat. Nos. 6,911,220; 6,306,651; 6,412,492; 6,514,513; 6,558,663; and 6,296,846. See also Kuwaki et al., Nature Med., 11(1):29-31, 2005, and Yamada et al., Nature Med. 11(1):32-34, 2005.

### EXEMPLIFICATION

### Example 1

### Materials and Methods

Animals

All studies were performed under an institutionally approved animal protocol in accordance with the NIH Guide. Female C57BL/6 (H-2^{b}), B10.A (H-2^{a}; L^{d+}), A.SW (H-2^{s}; L^{d-}), B10.S (H-2^{s}; L^{d-}) and B10.RIII (H-2^{r}) mice were purchased from Frederick Cancer Research Center (Frederick, Md.) or from The Jackson Laboratory (Bar Harbor, Me.). C57BL/6-2C-TCR transgenic (TCR-Tg) (H-2^{b}), C57BL/6-PD-1 KO (H-2^{b}) and C57BL/6-PD-L1 KO mice were bred in an animal facility. All mice were housed in a specific pathogen-free microisolator environment.

Conditioning

Age-matched (7-14 weeks old) mice received a low dose of TBI (3Gy) from a ¹³⁷Cesium irradiator on Day −1 with respect to BMT. When indicated, anti-CD8 mAb (2.43; 1.44 mg/mouse) was administered i.p. on Day-1. Blocking anti-PD-1 mAb (29F1A12, rat IgG2a, 200 μg/mouse), blocking anti-PD-L1 mAb (10F9G2, rat IgG2b, 200 ng/mouse), irrelevant rat IgG2a or rat IgG2b mAbs (200 ng/mouse) (BioExpress) were administered i.p. on days −1, 2, 5, 8 and 11 with respect to BMT. Anti-mouse CD154 mAb (MR1; 2 mg/mouse; National Cell Culture Center) was administered i.p. on Day 0 prior to transplantation with 20−25×10⁶ allogeneic BMC by tail vein injection. C57BL/6 mice with a traceable donor-reactive transgenic CD8 T cell (2C TCR-Tg CD8 cells) population were prepared as previously described (Fehr et al., Eur J. Immunol. 2005; 35:2679-2690) (FIG. 1A). These mixed 2C/wild-type C57BL/6 chimeras are referred to as 2C/B6 mice.

Flow Cytometric Analysis

Multilineage Chimerism in White Blood Cells

Four-color flow cytometric (FCM) analysis was performed on white blood cells (WBC) to analyze the development of multilineage chimerism (Tomita et al., Transplantation. 1996; 61:469-477). Donor-derived cells were identified in the live cell population (propidium iodide negative) using fluorescein isothiocyanate (FITC)-conjugated anti-H-2D^{d} mAb 34-2-12. Cells were counterstained with phycoerythrin (PE)-conjugated anti-CD4 (Becton Dickinson (BD)/PharMingen, San Diego, Calif.), or MAC-1 (Caltag, San Francisco, Calif.) and with Allophycocyanin (APC)-conjugated anti-CD8 or anti-B220 mAb (BD/PharMingen), respectively. Negative control mAbs included HOPC1-FITC (prepared in our laboratory) and rat anti-mouse IgG2a-PE or -APC. A mouse was considered chimeric when it demonstrated ≧5% of donor cells in all lineages tested.

Activation Markers on Splenocytes

2C CD8⁺ as well as non-2C CD8⁺ T cells were analyzed by FCM on live splenocytes using an anti-clonotypic mAb 1B2 (specific for the 2C TCR, Kranz et al., Proc Natl Acad Sci USA. 1984; 81:7922-7926) revealed by FITC-conjugated anti-mouse IgG1 mAb and APC-conjugated anti-CD8. PE-conjugated anti-CD69, anti-CD25, anti-CD44 and anti-PD-1 mAbs (BD/PharMingen) were used to detect the surface expression of activation markers. Negative control mAbs included HOPC1-FITC and rat anti-mouse IgG2a-PE or -APC. 10⁴ CD8⁺ splenocytes were acquired for each analysis.

For the analysis of dendritic cells, spleens were flushed with 1 ml of warm collagenase D in RPMI then cut into small pieces and incubated for 30 min at 37° C. in 6% CO₂. The reaction was stopped by adding 10% EDTA. The small pieces of spleen were mashed, washed and RBC were then lysed in ACK. The enriched DC population was stained with 34.2.12 FITC, anti-PD-L1 PE (BD/PharMingen) and anti-CD19 APC (BD/PharMingen) or anti-CD11c APC (BD/PharMingen). Negative control mAbs included HOPC1-FITC and rat anti-mouse IgG2a-PE or -APC.

Cell Mediated Lympholysis (CML) Assay

CML assay was performed as described (Ito et al., J. Immunol. 2001; 166:2981). Briefly, responders and stimulators were cocultured at a 1:1 ratio for five days. Cells were then serially diluted and co-incubated with ⁵¹Cr-labelled ConA blast target cells for four hours.

Statistical Analysis

Statistical analysis was performed using the Mann-Whitney U test. P values less than 0.05 were considered to be significant.

### Example 2

### Prolonged Upregulation of Activation Markers Upon Specific Antigen Recognition by CD8 T Cells Tolerized In Vivo

To specifically track donor-reactive CD8⁺ T cells in mice receiving allogeneic BMT, mice were generated which contain a majority of wild-type cells and a minority of traceable donor-reactive CD8 cells expressing the transgenic 2C TCR, which recognizes the MHC class I molecule L^{d} (FIG. 1A) (Sha et al., Nature. 1988; 335:271-274). The expression level of activation markers on 2C CD8⁺ T cells in 2C/B6 mice that received 3Gy TBI and 5×10⁶ 2C BMC 7 weeks prior to conditioning (3Gy TBI on Day-1, 2 mg anti-CD154 i.p.) followed by either L^{d+} (B10.A; H-2^{a}) or L^{d-} (A.SW or B10.S; H-2^{s}) BMT was compared. A 2C/B6 control group received TBI and L^{d+} BMT without anti-CD154. Such treatment does not permit development of chimerism (Takeuchi et al., Am J. Transplant. 2004; 4:31-40). 2C CD8⁺ cells are rapidly deleted within 2 weeks after BMT in recipients of L^{d+} BMT with this tolerizing regimen (Fehr et al., Eur J. Immunol. 2005; 35:2679-2690). However, 4 and 7 days after allogeneic BMT, the spleen contained a measurable 2C CD8⁺ population (FIG. 1B). The percentage of 2C CD8⁺ cells was much lower in the chimeric (B10.A BMT) than in the rejecting mice at both time points (FIG. 1B). By day 7, rejecting animals showed evidence of expansion of 2C cells, while tolerant animals showed partial deletion and recipients of irrelevant marrow contained near baseline 2C levels (FIG. 1B). By 2 weeks post-BMT, tolerant animals showed complete deletion of 2C alloreactive CD8 cells in the WBC (FIG. 1C). FIG. 2A shows that 2C CD8⁺ cells expressed activation markers (i.e. CD69, CD25, CD44) only when they were exposed in vivo to relevant L^{d+} BM and not when they were exposed to irrelevant L^{d-} marrow. The levels of these activation markers on 2C and non-2C CD8⁺ cells were negligible prior to allogeneic BMT, as shown in FIG. 2B. Surprisingly, tolerizing treatment with anti-CD154 mAb in mice receiving relevant L^{d+} BM did not prevent the activation of donor-reactive 2C CD8⁺ cells. As shown in FIG. 2A, similar levels of CD69, CD25 and CD44 activation markers were detected on 2C CD8⁺ cells 4 days after L^{d+} BMT, regardless of whether or not the recipient was treated with anti-CD154. However, CD69 and CD25 were no longer detectable 7 days after BMT on 2C CD8 cells from rejecting animals not treated with anti-CD154, while CD44 was still highly expressed. In contrast, high levels of expression of CD69, CD25 and CD44 were maintained 7 days after BMT on tolerized 2C CD8⁺ cells (FIG. 2A). This prolonged upregulation of activation markers in the tolerized population is associated with the persistence of donor antigen, which disappears in the control mice due to the rejection of donor BMCs (data not shown).

The non-2C polyclonal CD8⁺ population in tolerant animals (i.e., recipients of the full conditioning regimen and L^{d+} or L^{d-} BMT) did not show any increase in CD69 or CD25 activation markers (FIG. 2A). However, polyclonal CD8 T cells (i.e., non-2C CD8 cells) in mice receiving BMT without anti-CD154 treatment expressed significantly higher levels of CD69, CD25 and CD44 four days after BMT compared to mice receiving BMT with the tolerizing regimen. The increased polyclonal CD8 activation on Day 4 in animals receiving BMT without anti-CD154 is consistent with the rejection process, which not only involves 2C CD8⁺ T cells but also polyclonal CD8 cells in the 2C/B6 recipients. Since polyclonal donor-reactive CD8 cells, like the 2C cells (FIGS. 1B-C), survived and/or expanded in the non-tolerant group when they were presumably beginning to be deleted in the tolerant group (like 2C in FIGS. 1B-C), there is an increase in the total percentage of activated polyclonal CD8 cells in the non-tolerant compared to the tolerant group on Day 4 (FIG. 2A). By 7 days, when animals receiving BMT without anti-CD154 mAb had rejected their grafts, polyclonal CD8 cells still expressed high levels of CD44 but had lost the CD69 and CD25 activation markers (FIG. 2A).

### Example 3

### Upregulation of PD-1 Upon Specific Antigen Recognition In Vivo by Tolerized and Non-Tolerized CD8 T Cells

The level of PD-1 expression on 2C and non-2C CD8⁺ T cells was investigated 4 and 7 days after BMT. PD-1 was found to be highly expressed on 2C CD8⁺ cells in mice receiving L^{d+} relevant allogeneic marrow, regardless of whether or not they were treated with anti-CD154 (FIG. 2A). PD-1 expression on 2C CD8⁺ cells was significantly greater when mice received the tolerizing regimen with L^{d+} relevant BM than in mice receiving the irrelevant control marrow (FIG. 2A), indicating that PD-1 upregulation is antigen-driven. PD-1 expression was significantly greater on non-2C CD8⁺ cells 7 days after L^{d+} BMT without anti-CD154 treatment (i.e., in rejecting mice) than in tolerized mice (FIG. 2A), consistent with the expansion of activated cells in rejecting mice (FIGS. 1B-C).

### Example 4

### Tolerized CD8 T Cells are in an Abortive Activation State

Despite persistently high levels of activation markers on donor-specific CD8 cells and expression of the inhibitory PD-1 receptor, these animals accepted allogeneic BM when treated with anti-CD154. It was hypothesized that these CD8 T cells were already tolerized within the first week post-BMT. Consistent with this interpretation, FIG. 3A shows that restimulated CD8 cells obtained from 2C/B6 mice receiving L^{d+} BM with anti-CD154 were unable to kill donor target (B10.A) cells, while maintaining CTL activity against third party cells 4 days after allogeneic BMT. As shown in FIG. 3B, 2C/B6 mice receiving L^{d-} BM (B10.S) with anti-CD154 were not tolerant to B10.A targets and therefore were able to kill B10.A as well as targets from another allogeneic strain (B10.RIII). Finally, 2C/B6 mice receiving L^{d+} BM without anti-CD154 were not tolerant to the B10.A marrow, as they were able to kill donor target (B10.A) cells (FIG. 3C). Thus, donor-reactive CD8⁺ cells from chimeras underwent abortive activation that culminated in effector-CTL tolerance within 4 days of BMT with anti-CD154. Similar results were obtained when splenocytes were analyzed 8 days after BMT (data not shown).

### Example 5

### The PD-1/PD-L1 Pathway is Essential for CD8 but not CD4 Tolerance in Recipients of Allogeneic BMT with Anti-CD154

The requirement for the PD-1 pathway was compared in two similar models in which the only difference is that only peripheral CD4 (and not CD8) T cell tolerance is required in one model because CD8 cells are depleted with mAb. While wild-type (WT) control mice successfully achieved multilineage mixed chimerism with or without CD8 depletion, PD-1 KO mice failed to develop mixed chimerism unless the recipients were depleted of CD8 cells (FIG. 4A-Table 1). The failure to achieve even initial engraftment of B10.A marrow in PD-1 KO mice receiving anti-CD154 and 3Gy TBI while chimerism was achieved in CD8-depleted PD-1 KO mice suggested that PD-1 may be required for the peripheral tolerance of CD8 but not CD4 T cells. To further address the role of the PD-1 pathway, marrow engraftment was evaluated in wild-type recipient mice treated with blocking mAbs targeting PD-1 and PD-L1. Blocking the PD-1 pathway with these mAbs prevented the development of mixed chimerism in 7 of 8 mice that were not depleted of CD8 cells (FIG. 4B, Table 2). In contrast, blocking mAbs against the PD-1 pathway did not impair the establishment of multilineage mixed chimerism in mice that were initially depleted of CD8 T cells. When mAbs against either the PD-L1 or the PD-1 molecule were used in recipients of BMT, 3Gy TBI and anti-CD154, allogeneic BMCs were rejected, whereas mice receiving control mAb showed a high incidence of mixed chimerism (FIG. 4B). Therefore, the PD-1/PD-L1 pathway is critical in tolerizing alloreactive CD8 and not CD4 T cells in this model.

TABLE 1
Multilineage chimerism 6 weeks after BMT in mice from the experiment
presented in FIG. 4A.

| | | | | Mean ± SD % donor chimerism | | |
|---|---|---|---|---|---|---|
| | Anti-CD8 | | | among chimeric mice | | |
| # in group | mAb | Recipients | # of chimeras | Mac1⁺ Cells | B Cells | CD4⁺ Cells |
| 7 | Yes | C57BL/6 | 7 | 60.06 ± 20.69 | 48 ± 17.07 | 31.23 ± 12.27 |
| 8 | Yes | C57BL/6 PD-1^{-/-} | 8 | 33.75 ± 9.27 | 51.86 ± 4.16 | 26.33 ± 4.63 |
| 7 | No | C57BL/6 | 6 | 60.62 ± 10.15 | 59.42 ± 7.67 | 32.47 ± 4.49 |
| 5 | No | C57BL/6 PD-1^{-/-} | 0 | 0 | 0 | 0 |

TABLE 2
Multilineage chimerism 6 weeks after BMT in mice from the experiment
presented in FIG. 4B.

| | | | | Mean ± SD % donor chimerism | | |
|---|---|---|---|---|---|---|
| # in | Anti-CD8 | Anti PD-1 and | # of | among chimeric mice | | |
| group | mAb | anti-PD-L1 mAb | chimeras | Mac1+ Cells | B Cells | CD4+ Cells |
| 7 | Yes | No | 7 | 76.8 ± 11.69 | 55.55 ± 22.09 | 31.10 ± 5.33 |
| 8 | Yes | Yes | 8 | 61.15 ± 24.17 | 62.17 ± 11.96 | 38.04 ± 9.08 |
| 7 | No | No | 6 | 59.02 ± 17.34 | 59.47 ± 11.31 | 36.03 ± 5.92 |
| 8 | No | Yes | 1 | 64.19 | 46.33 | 9.10 |
| 8 | No | Anti-PD-L1 mAb alone | 1 | 6.3 | 7.57 | 17.78 |
| 5 | No | Anti-PD1 mAb alone | 1 | 5.82 | 30.16 | 14.88 |

### Example 6

### CD4 Depletion Impairs Upregulation of PD-L1 on APC

Recipient B cells and dendritic cells (DC) are needed for CD8 tolerance in this protocol. Since CD4 cells and PD-L1 are also involved in CD8 tolerance, the effect of CD4 cells on PD-L1 expression on recipient B cells (CD19+) and dendritic cells (CD11c+) was analyzed in mice that received B10.A BMT with our regimen, with or without anti-CD4 treatment. Mice receiving BMT without anti-CD154 showed a significant increase in PD-L1 expression on DC relative to mice receiving anti-CD154. A lesser, statistically insignificant increase was observed on B cells of mice receiving no MR1 (FIG. 5A). These data suggest that recipient DC were activated to a greater extent when anti-CD154 treatment was omitted from the conditioning regimen. On the other hand, when fully conditioned recipients were also CD4 depleted, PD-L1 expression on both DCs and B cells was significantly lower than when CD4 cells were not depleted (FIG. 5B). These results suggest that, despite blockade of the CD154/CD40L interaction, CD4 T cells still provide some activating signal to recipient APCs, allowing sufficient up-regulation of PD-L1 for interactions with PD-1 on CD8 T cells that promote CD8 tolerance.

### Example 7

### Cell-Intrinsic Requirement for PD-1 on CD8 T Cells

CD8 T cell-deficient MHC class I-null (K^{b}D^{b} KO) animals were treated with 3 GY TBI on day −1 and 2 mg anti-CD154 on day 0, followed by i.v. injection of 9×10⁶ adoptively transferred CD8 T cells from either wild-type B6 animals (WT) or PD-1 KO animals and 25×10⁶ T cell-depleted B10.A (fully MHC-mismatched) BMCs. (FIG. 6A) shows the percent donor chimerism in the B220+ lineage (representative of all lineages) of the peripheral blood for each individual animal (FIG. 6B) shows the average and standard deviation of peripheral B220 chimerism for both groups. (FIG. 6C) shows the incidence of chimerism at 7 weeks post-BMT.

The data herein show a critical role for the PD-L1 molecule in tolerizing alloreactive CD8 T cells in vivo. When CD8 T cells first encounter alloantigen in the absence of CD40-mediated signals to APC, activation of the PD-1 pathway by PD-L1 on the APC may impair the proliferation, cytotoxic differentiation, cytokine production and survival of allospecific CD8 T cells. Alloreactive CD8 T cells have been shown to prevent tolerance induction in primates and humans receiving costimulatory blockade. Therefore, activating the PD-1 pathway provides a new strategy to specifically promote tolerance of alloreactive CD8 T cells.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. A method of promoting acceptance, by a recipient mammal, of a graft from a donor mammal, the method comprising:
administering to the recipient, a Programmed Cell Death 1 (PD-1) agonist; and
introducing into the recipient mammal, hematopoietic stem cells.

2. The method of claim 1, wherein the PD-1 agonist comprises a Programmed Cell Death 1 Ligand 1 (PD-L1) polypeptide, or a biologically active fragment thereof.

3. The method of claim 2, wherein the PD-1 agonist comprises PD-L1-Ig.

4. The method claim 1, wherein the PD-1 agonist comprises a soluble Programmed Cell Death 1 Ligand 2 (PD-L2) polypeptide, or a biologically active fragment thereof.

5. The method of claim 4, wherein the PD-1 agonist comprises PD-L2-Ig.

6. The method of claim 1, wherein the PD-1 agonist is administered prior to administering the hematopoietic stem cells.

7. The method of claim 1, wherein the PD-1 agonist is administered immediately prior to, or at the same time as, the hematopoietic stem cells.

8. The method of claim 1, wherein the PD-1 agonist is administered after the hematopoietic stem cells.

9. The method of claim 1, further comprising administering an inhibitor of a costimulatory pathway.

10. The method of claim 9, wherein the inhibitor of the costimulatory pathway is administered immediately prior to, or at the same time as, the hematopoietic stem cells.

11. The method of claim 9, wherein the inhibitor of the costimulatory pathway is administered after the hematopoietic stem cells.

12. The method of claim 9, wherein an inhibitor of CD40-CD154 interaction is administered.

13. The method of claim 12, wherein the inhibitor of the CD40-CD154 interaction comprises an anti-CD154 antibody.

14. The method of claim 12, wherein the inhibitor of the CD40-CD154 interaction comprises an anti-CD40 antibody.

15. The method of claim 9, wherein an inhibitor of CD28-B7 interaction is administered.

16. The method of claim 1, further comprising implanting a graft in the recipient mammal.

17. The method of claim 16, wherein the graft is implanted prior to administering the hematopoietic stem cells.

18. The method of claim 16, wherein the graft is implanted at the same time as the hematopoietic stem cells.

19. The method of claim 16, wherein the graft is implanted after the hematopoietic stem cells.

20. The method of claim 16, wherein the graft comprises a heart, liver, or kidney.

21. The method of claim 1, wherein the donor mammal is the same species as the recipient mammal.

22. The method of claim 1, wherein the donor mammal is a different species than the recipient mammal.

23. The method of claim 1, wherein the recipient mammal is a human.

24. The method of claim 1, wherein the method comprises administering an anti-T cell antibody to the recipient mammal.

25. The method of claim 24, wherein the anti-T cell antibody comprises an anti-CD2 antibody.

26. The method of claim 1, wherein the method comprises administering an immunosuppressive agent to the recipient mammal.

27. The method of claim 26, wherein the immunosuppressive agent comprises cyclosporine A.

28. The method of claim 1, wherein the method comprises administering non-myeloablative irradiation or a non-myeloablative chemotherapeutic treatment to the recipient mammal, prior to administering the hematopoietic stem cells.

29. The method of claim 28, wherein the non-myeloablative chemotherapeutic treatment comprises treatment with cyclophosphamide.

30. The method of claim 1, wherein the amount of hematopoetic stem cells introduced into the recipient is sufficient to induce chimerism in the recipient.

31. The method of claim 1, wherein the method promotes acceptance of the hematopoietic stem cells.

32. The method of claim 16, wherein the graft and the hematopoietic stem cells are from the same donor mammal.

33-34. (canceled)

35. A method of promoting acceptance, by a recipient mammal, of a graft from a donor mammal, the method comprising:
administering to the recipient, a chemotherapeutic agent;
administering to the recipient an anti-T cell antibody;
administering to the recipient thymic irradiation;
administering to the recipient, a PD-1 agonist;
administering to the recipient; cyclosporine A;
introducing into the recipient mammal, hematopoietic stem cells; and
implanting a graft into the recipient.

36. The method of claim 35, wherein the chemotherapeutic agent comprises cyclophosphamide.

37. The method of claim 35, wherein the chemotherapeutic agent comprises an anti-CD2 antibody.

38. The method of claim 35, wherein the PD-1 agonist comprises PD-L1-Ig.
